(19) United States
(12) Patent Application Publication
(10) Pub. No.: US 2008/0214940 A1
(43) Pub. Date: Sep. 4, 2008

(54) MEDICAL IMAGING LENS SYSTEM, AND METHOD WITH HIGH-EFFICIENCY LIGHT COLLECTION AND COLLINEAR ILLUMINATION

(76) Inventors: DAVID A. BENARON, Portola Valley, CA (US); Illian H. Parachikov, Belmont, CA (US)

Correspondence Address: MORGAN, LEWIS & BOCKIUS, LLP., 2 PALO ALTO SQUARE, 3000 EL CAMINO REAL, PALO ALTO, CA, 94306, US

(21) Appl. No.: 12/040,881

(22) Filed: Mar. 1, 2008

Related U.S. Application Data
(60) Provisional application No. 60/904,591, filed on Mar. 2, 2007.

(51) Int. Cl. A61B 6/00

(52) U.S. Cl. 600/478

(57) ABSTRACT

We have discovered an improved lens system for biomedical optical imaging applications for collecting light from tissue with an improved efficiency and geometry, and for delivering collinear, pre-aligned illumination to a the sample, for the purpose of enabling imaging applications in which a catadioptric lens and mirror system (103) is used for light collection, and integrated collimating illumination optics and aperture (159) have been provided, has been constructed in accordance with the present invention to allow for high-efficiency light collection in operating room and radiology suite imaging geometries. The efficient collection of light from the collinear illumination, allows this lens system to operate at higher speeds and with improved ease-of-use, as well as to be integrated into a lens system (103), a medical probe (255), or a catheter (270). A medical system incorporating the improved device, and medical methods of use, are described.

### CROSS REFERENCE OF RELATED APPLICATIONS

This application claims the benefit of and priority to U.S. Provisional Patent Application No. 60/904,591, entitled “Biomedical Imaging Lens and Systems with High-Efficiency Light Collection and Collinear Illumination”, filed Mar. 2, 2007, the disclosure of which is incorporated herein by reference in its entirety.

This invention was made with United States Government support, contract nos. CA-083597, CA-88190, and CA-107908 awarded by the U.S. National Institutes of Health. The Government has certain rights in the invention.

### FIELD OF THE INVENTION

The present invention relates generally to imaging devices and systems for providing a high efficiency of light delivery to biological organisms, tissues, or agents, and more particularly relates to the embedding of collinear illumination optics within a high-light-collection, low-numerical aperture lens of catadioptric design for the purpose of performing real-time in vivo planar or tomographic optical imaging of living animals, thus avoiding some of the weight, cost, risk, alignment, and quantitation limitations inherent in conventional imaging lens or mirror systems which lack such high efficiency light collection and/or have separate illumination optics.

### BACKGROUND OF THE INVENTION

The standard method for collecting low-level light from fluorescent, chemiluminescent, or bioluminescent systems is to provide for a standard lens, coupled to a solid state imaging detector such as a cooled or intensified CCD. Such systems are needed as light from living tissue is released in a scattered manner, and thus requires either direct coupling (e.g., fiber to tissue) or a focusing system (e.g., a lens) in order to form an image. However, such traditional light sources have significant native disadvantages, including that: (a) they tend to either be macro focus (focus at millimeters from a millimeter-wide subject) or zoom (focus many meters away and a large object), and focus poorly upon a surgical field that may be of intermediate size (i.e., 5-20 cm in diameter) and of intermediate distance (i.e., 50-100 cm from the lens), (b) they collect their light rather inefficiently, with a focus on spatial resolution above light-gathering, and (c) if the biological process being imaged requires light (such as a fluorescence process), they tend to have no seamlessly integrated method for providing light to the subject, and thus whenever the lens is moved, the light source must be separately readjusted and aligned.

These limitations are best appreciated by example.

First, with specific regard to focus, most lenses tend to fall into three categories: wide angle, which collects a small portion of the light from many angles, telephoto, which collects as much as possible from a small area focused far away, or macro-zoom, which allows focus of the lens down to a few millimeters or centimeters from the subject. None of these lenses are optimized to focus and image an area 5-30 mm across from 0.5 to 2 m away.

Second, with respect to light gathering, optical contrast reporters tend to be weak emitters, and these emitters typically produce light in all directions—that is, relatively uniformly over a full 4π spherical angle—in the absence of mirrors or lenses. This broad spatial emission typically makes the optical coupling of light from an optical contrast agent into a distant lens very inefficient. For illustration, consider a 1 cm diameter lymph node, stained with an optical contrast agent. A lens is placed 1 meter from the lymph node. For this node, the light emitted can be considered as illuminating the inner surface of a sphere 2 meters in diameter, with the lymph node at the center. This sphere has an inner surface area of 4/3*π*r², or 4.2 million mm². However, only a portion of this light reaches the lens. For a typical lens with an aperture of 28 mm (e.g., Nikon c-mount), the area of the lens is 2*π*r, or 88 mm². The lens then samples only (88/4,200,000) of the light emitted by the lens, for a sampling efficiency of only 0.0021%. That is, the lens intercepts only a tiny portion of the uniform field of radiated light with 99.9979% of the fluorescent contrast signal wasted. This makes for a very inefficient imaging, and for weak detection that requires strong signals in order to be detected.

Third, co-illumination is important in applications in which a signal is produced in response to illumination. A typical illumination setup requires adjustment of the illuminating light to cover a particular region of the subject or tissue, followed by adjustment of the imaging camera. In a surgical procedure, this is not acceptable. The surgeon may wish to point the camera in multiple directions, and desires to have the image and the illumination move in synchrony.

Last, when attempting to combine an imaging or detection with feedback to a therapeutic, a lack of co-illumination makes it difficult to get real time feedback. In contrast, co-illumination facilitates co-registration of the detected signal with an illumination used as a therapeutic. In this manner, for example, a detected signal could trigger an increase in the power of light to that region, allowing photodynamic or other light sensitive therapies to be selectively triggered in the same region that provided the detection signal, thus coupling the imaging to a therapeutic method.

These limitations of conventional lenses are apparent in the art. Catadioptric lenses are known in the art, and some of these are suggested for certain medical or biomedical uses (e.g., U.S. Pat. No. 5,095,887 as a surgical endoscope, U.S. Pat. No. 5,490,849 for delivery of light during corneal ablation, U.S. Pat. No. 6,256,143 for viewing by eye during stereoscopic microscopy). However none are suggested for biomedical optical imaging as combination light sources and imaging lenses, and their high-light collection and the option of co-illumination have not been cited nor exploited for biomedical optical imaging purposes, especially in medicine for in vivo uses in the operating room or radiology suite.

Various schemes for illumination or for transmitting light to an imaging sample are known (e.g., such as light conducting rods in U.S. Pat. No. 5,974,210), but none with the purpose of improving the collinear efficiency of delivery, nor are there lenses specifically designed to operate as an integrated illuminators with high delivery efficiency. Examples of invasive or tissue surface monitoring devices equipped with illumination optics include catheters, needles, and trocars (e.g., U.S. Pat. No. 5,280,788, U.S. Pat. No. 5,931,779), as well devices containing the light source itself (e.g., U.S. Pat. No. 5,645,059, U.S. 5,941,822, WO 00/01295). These systems typically completely ignore the complex issues of illumination source design, suggesting only that known or existing light sources can be used rather than proposing improved illumination sources, and none of these systems consider specifically design issues regarding design and collinear deployment of optical light sources, especially with regard to imaging.

Therefore, all of the above focusing and illumination systems and methods suffer from one or more limitations noted above, in that they function poorly at distances used for the imaging of living subjects, they are not designed for imaging, they collect light poorly, they are not configured to deliver collinear light with a high efficiency, and/or they ignore or omit design considerations regarding lens design and illumination efficiency, and thus fail to reliably provide an improved focusing and illumination source for use in real-time, biomedical optical imaging of living tissue.

None of the above systems suggest or teach a method and system to more efficiently collect and focus light from living tissue or spectroscopy samples, and if needed to provide a highly-efficient, collinear illumination for the performance of biomedical optical imaging in living samples. A collection-and-delivery-optimized light focusing device and system has not been taught, nor has such a tool been successfully commercialized.

### SUMMARY OF THE INVENTION

The present invention relies upon the knowledge of the design considerations needed to achieve a high-throughput catadioptric imaging lens, with the option of high-efficiency and collinear light delivery.

In one aspect, the present invention provides an imaging system comprising an illumination element and light collection element comprising a catadioptric lens, wherein the illumination element and the light collection element are arranged so as to provide a substantially co-registered illumination and imaging plane. In some embodiments, the illumination element comprises a light input port and an illumination aperture optically coupled to the light input. In some embodiments, the illumination aperture is optically couple to the light input port through a beam expander.

In some embodiments, the light collection element comprises a proximal entrance aperture, a proximal lens, a first mirror optically coupled to said entrance aperture through said proximal lens, a second mirror optically coupled to said first mirror, and a distal exit aperture optically coupled to said second mirror. In some embodiments, the imaging system further comprises a first polarizing filter and a second polarizing filter. The first polarizing filter and the second polarizing filter are placed in parallel or with cross-axis. In some embodiments, the imaging system further comprises a bandpass filter. In some embodiments, the imaging system comprises a housing comprising a proximal half and a distal half joined at a sliding joint. In some embodiments, the imaging system comprises an angle dependent filter, such as an interference filter, or a notch filter.

In one aspect, the present invention provides a method of obtaining an image of a subject, the method comprising: projecting light to a subject using an illumination element; and obtaining an image of the subject by collecting light from the subject with a light collection element, wherein the light collection element comprising a catadioptric lens, and wherein the illumination element and the light collection element are arranged so as to provide a spatially co-registered illumination and imaging plane. In some embodiments the illumination element and the light collection element are arranged to provide collinear illumination.

In some embodiments there is provided a focusing and imaging lens and illuminator for use in performing optical imaging on samples or living animals, with a higher throughput efficiency than with conventional lens sources, and with the option of collinear illumination, for the purpose of real-time biomedical optical imaging. In one example, the imaging system uses catadioptric lens and mirror system with a central collimated delivery beam, which can then be transmitted through free space to a sample, such as a target tissue, resulting in a high efficiency delivery of light to the target tissue and a high-throughput collection. The efficient collection of light and the collinear, pre-aligned illumination combine to allow this focusing and illuminator system to be integrated into biomedical optical imaging equipment, and deployed in the research lab, the radiology center, or the surgical suite. Medical systems incorporating the improved illuminator and medical methods of use are also described.

### BRIEF DESCRIPTION OF THE DRAWINGS

The following drawings are provided:

FIG. 1 is a schematic diagram of lens and illumination system constructed in accordance with one embodiment of the present invention.

FIGS. 2A to 2F show the improved illuminator of FIG. 1 as incorporated into alternative embodiments of the present invention.

FIG. 3 shows an image collected using the system described in FIG. 1.

FIGS. 4A and 4B show images collected in an animal using the system described in FIG. 1.

### DETAILED DESCRIPTION OF THE INVENTION

### I. Definitions

For the purposes of this invention, the following definitions are provided:

Anterior/Posterior: The side closest to the source light is Anterior, while the side farthest from the light is Posterior. For example, the entrance for light in the lens system is Anterior, whereas the point of attachment of the lens system to the camera is Posterior.

Catadioptric: A folded-path lens system combining mirrored reflective surfaces as well as diffractive transmitting lenses such that the linear distance traveled by the signal inside the lens is much greater than the physical length of the exterior of the lens itself. An example of a catadioptric lens, without limitation, is a Cassegrain Catadioptric telescope.

Collinear: A system in which the axis of illumination and the axis of imaging are equivalent or substantially and effectively equivalent. This word is equivalent to the hyphenated word “co-linear”.

Coregistered: A system in which the area of illumination and the area for imaging are substantially overlapping and/or effectively equivalent in specific area covered. This word is equivalent to the hyphenated word “co-registered”.

Efficiency: As used herein, the percentage of the total usable light that can be coupled to the imaging site, and then returned to the imaging system for collection, processing, and analysis.

Field of View: The region effectively imaged using the light collection system.

Field of Illumination: The region illuminated using the light collection system.

Illumination Plane: The surface illuminated by the illuminating light source. Typically, this surface will be more or less grossly perpendicular to the surface to be illuminated, so that the illuminating lamp can cast light upon this surface. For example, during surgery on the prostate, the open abdominal cavity would be in the illumination plane, and for the purposes of this invention, this would be co-registered with the imaging plane of the camera suspended above the operating field.

Imaging Plane: The surface imaged by the camera. Typically, this surface is more or less grossly perpendicular to the imaging camera, so that the imager can be focused on the imaging plane. For example, during surgery on the prostate, the open abdominal cavity is the imaging plane for a camera suspended above the operating field.

In Vivo: A measurement performed on tissues on or within a living animal, plant, viral, or bacterial subject.

Light: Electromagnetic radiation from ultraviolet to infrared, namely with wavelengths between 10 nm and 100 microns, but especially those wavelengths between 200 nm and 2 microns, and more particularly those wavelengths between 550 and 1650 nm, wavelengths effectively transmitted by tissue without damage, and wavelengths efficiently absorbed and then returned as fluorescence by in vivo fluorescent contrast agents.

Light Detector: A detector that generates a measurable signal in response to the light incident on the detector. In this system, the light imaging system is coupled variably to an intensified CCD, a cooled CCD, though other detectors could be substituted.

Light Source: A source of illuminating photons. It may be composed of a simple light bulb, a laser, a flash lamp, an LED, a white LED, or another light source or combination of sources, or it may be a complex form including, a light emitter such as a bulb or light emitting diode, a collection of LEDs or lamps, one or more filter elements, a transmission element such as an integrated optical fiber, a guidance element such as a reflective prism or internal lens, and other elements intended to enhance the optical coupling of the light from the source to the tissue or sample under study. The light may be generated using electrical input (such as with an LED), optical input (such as a fluorescent dye in a fiber responding to light), or any other source of energy, internal or external to the source. The light source may be continuously on, pulsed, or even analyzed as time-, frequency-, or spatially-resolved. Time and frequency analysis may require a pulsed or modulated light source, and time or frequency sensitive detection systems. A pulsed laser, such as a Coherent Tsunami or other pulsed laser works well for time-resolved methods, as well as for brief collection periods that permit reductions in the influence of room light in the collection. The light emitter may consist of a single or multiple light emitting elements, such as a combination of different light emitting diodes to produce a spectrum of light.

Optical Coupling: The arrangement of two optical elements such that light exiting the first element interacts, at least in part, with the second optical element. This may be free-space (unaided) transmission through air or space, or may require use of intervening optical elements such as lenses, filters, fused fiber expanders, collimators, concentrators, collectors, optical fibers, prisms, mirrors, or mirrored surfaces. For most optical elements, there is an entry end, where light enters, and an exit end, where light exits. This can also be defined as a proximal end nearest the light source, and a distal end nearest the sample. These two descriptions are not equivalent, for example the proximal end of an optical fiber may be the entry or the exit end, depending on whether light in the fiber is traveling toward or away from a sample region.

Real Time: A measurement performed in a few minutes or less, and preferably in under 1 to 10 seconds. In medical or surgical use, such real-time measurements allow a procedure or a treatment plan to be modified based upon the results of the measurement.

### II. The System

In general, a collinear illumination and imaging system having an illumination element and a light collection element is provided. The collinear illumination element and the light collection element are arranged so as to provide a spatially co-registered illumination and imaging plane. In a preferred embodiment, the light collection element is comprised of a catadioptric lens, which by design allows the illumination element and the light detection element to be arranged to provide collinear illumination and light collection.

In one aspect of the present invention, an optical lens and system is provided. In one embodiment, the invention provides a high-efficiency catadioptric optical lens with collinear illumination and imaging paths within an assembly comprising a central light source optical element. By “high efficiency” herein is meant a low-NA lens, in this case f 0.7 to f 1.0, but at least less than f 4.0, which focuses over distances larger than typical macro lens (e.g., more than 10 cm) but far closer than typical telephoto (e.g., less than 3 m).

Embodiments of the improved focusing and imaging lens and illuminator as described in the teachings of the present invention has multiple advantages as described herein. For example, embodiments of the present invention provide an improved illumination density, distribution, and/or efficiency, as compared to a separately-adjusted, non-integral light delivery system, if required.

In one aspect, the imaging system provided herein comprises an illumination element comprising a light input port and an illumination aperture, said aperture optically coupled to the light input port. The light input port or light entrance port is focused or directed by movement of the lens, without a required independent movement of the imager lens and an illumination system.

In some embodiments, the illumination aperture is optically coupled to a beam expander. By “beam expander” herein is meant a device that expands a small beam, such as that contained in a delivery fiber, to a substantially parallel or collinear expanded beam that moves and re-points to a new illumination and imaging area with lens movement.

In some embodiments, the illumination element is a central light source optical element. This advantageous central light source allows the illumination to be pointed at the target within the least efficient area of catadioptric collection (the center), while being sufficiently self-contained as to prevent internal reflections from the inner lens surface, or within the lens body and housing, which can be problematic in free-space transmission of illuminating radiation within a lens. In some embodiments, the central light source optical element, at least in part, is bounded by a central tube to further isolate the illumination radiation from the return path, and the source comprises a source input entrance port optically coupled through a beam expander to a source output exit aperture. As described herein in more details, having part of the light source optical element bound by a tube separates the illumination and collection path within the lens. The advantage of having the ability to provide separation of the illumination and collection paths within the lens is to reduce cross-talk within the lens, which can be difficult to achieve without specific design considerations, such as a central illumination barrel that protrudes to or beyond the outside rim of the lens housing. Such protrusion is an important improvement of the instant invention, and reduces internal reflections from the illumination source, while not in and of itself required for all devices constructed in accordance with the present invention.

In another aspect, the imaging system provided herein comprises alight collection element. In some embodiments, the light collection element comprises a catadioptric lens. While the collection of light using conventional lenses is typically poor in terms of efficiency, working distance, and focus, the collected light efficiency can be increased using a catadioptric configuration. An imaging lens with sufficiently improved light collection can substantially reduce the time required for imaging, in some cases allowing for real-time imaging during interventional procedures, or to allow detectability of previously undetectable image features, wherein use of conventional imaging lenses would have otherwise resulted in unacceptably long integration times.

In some embodiments, the light collection element comprises: a proximal entrance aperture; a proximal lens; a first mirror optically coupled to said entrance aperture through said proximal lens; a second mirror optically coupled to said first mirror; and a distal exit aperture optically coupled to said second mirror.

In some embodiments, a peripheral light collection optical element is provided. The light collection element has optical elements placed outside of a central tube, and a centrally-obscured anterior annular entrance aperture optically coupled through a proximal lens optically coupled to a posterior mirror optically coupled to an anterior mirror optically coupled to a final distal image exit plane. The peripheral element surrounds the central element over at least a portion of the length of the central element. The peripheral element is at least partially shielded from the central element by at least a portion of the central tube assembly, arranged so as to extend beyond said anterior annular entrance. The proximal annular entrance is thus optically isolated from said proximal source aperture. The central and peripheral elements are arranged so as to form a catadioptric lens for providing a spatially co-registered illumination and imaging plane external to the assembly.

In some embodiments, the illumination element and the light collection element are arranged so as to provide a spatially co-registered illumination and imaging plane. In some embodiments, the illumination element and said light collection element are arranged to provide collinear illumination.

Collinear illumination allows for a predictable light density and a light illumination area that moves when the lens is pointed in a new direction. This is important, as a surgeon (for example) is comfortable pointing an overhead lens in a new direction, but does not have the time or interest in alignment of a light source with this new illumination direction. Thus, the illumination is delivered with maximum efficiency, and being pre-aligned.

In another aspect, while the conventional systems require separate adjustment of the illumination and the imaging systems, a catadioptric system allows for collinear illumination, with co-adjustment of both the imaging and illumination systems with one re-pointing of the imaging system.

In another aspect, the same illumination system can be used to deliver light of a higher power, thus facilitating treatment (ablation, activation of photodynamic or other light-activated dyes) through the same lens that allowed for imaging, guidance, and even spectral analysis and tissue diagnosis, all as a direct result of collinearity of both the imaging and illumination systems.

Accordingly, in one aspect, embodiments of the present invention provide an optical focusing lens system, with an improved delivery efficiency over a conventional lenses,—that is, with an overall delivery imaging collection efficiency at least twice as good, and ideally 25 times or more better, than a typically achieved by a comparable conventional lens. A high-efficiency lens can have an f-number of 2 or less, or ideally 1.5 or less, or optimally 1.0 or less.

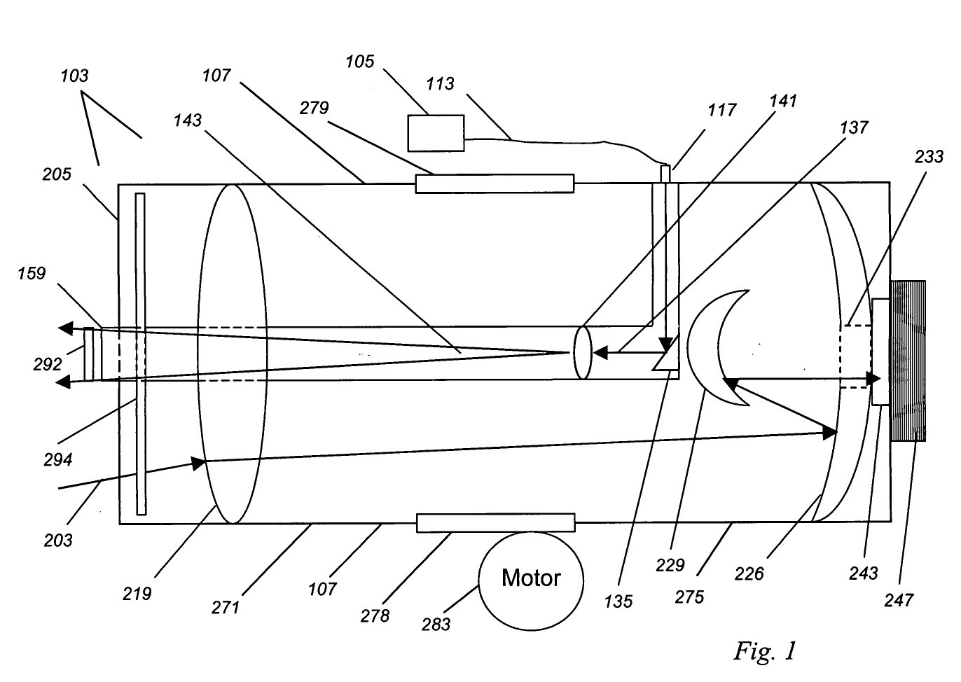

In some embodiments, the lens having collimated (parallel) ray sections. The advantage of having collimated (parallel) ray sections in the lens is to allow for specific bandpass or notch filters to be integrated within the lens. There are multiple methods of making such a parallel ray region, but typically this involves use of the appropriate lens selected such that imaged rays entering the lens are substantially parallel in the collimated region, and may be additionally refocused on a CCD detector after this region, or may be sufficiently focused so as to merely impinge on a CCD without additional focusing. Examples are shown in FIG. 1 as light ray 203 reflects off of mirror 229 and passes through mirror 226 traveling in a manner substantially parallel to other rays that may be traced from aperture 205 back to mirror 135. These filters can be motorized and automated (or electronically modulated) to allow for multiple filters to be used, such as for multiwavelength or hyperspectral analyses, or depth algorithms which use changes in the transmitted spectrum to determine depth of monitoring.

In some embodiments, the system comprises a first polarizing filter and a second polarizing filter. Polarizing filter or polarizer converts an unpolarized or mixed-polarization beam of light into a beam with a single polarization state (usually, a single linear polarization. In some embodiments, the first and the second polarizing filters are placed in parallel. In some embodiments, they are placed with cross-axes.

In some embodiments, the system comprises a bandpass filter. A bandpass filter passes frequencies within certain range and rejects (attenuates) frequencies outside that range. Such filters may be fixed filters, multiple filters of predetermined bandwidths and characteristics, or dynamic and tunable filters such as the Varispec from CRI (Woburn, Mass.).

In some embodiments, the system comprises an angle-dependent filter, such as an interference filter or a notch filter, which again can be fixed or dynamic, single or multiple homogenous or variable gradient.

In some embodiments, the imaging system comprises a housing and the housing comprises a proximal half and a distal half joined at a sliding joint. The sliding joint can be moved, such as by a motor. Moving the sliding joint changes the length of the housing and thus changes the focus of the lens. The movements of the two halves can be controlled by the motor, which can be further controlled by computer software. The computer software can have a distance-measuring function. Thus, the system will allow auto focusing or focusing by a user.

A variety of material can be used to build the housing, includes but is not limited to: metal, plastic and composite materials.

In another aspect, the present invention provides for focusing and imaging lenses with integrated illumination optics.

In some embodiments the light source is a laser selected so as to emit light which passes optimally through said proximal collection aperture to said distal focal imaging plane.

Alternatively, the light source is a laser selected so as to emit light which passes minimally through said proximal collection aperture to the distal focal imaging plane and which induces a fluorescent dye at or near said distal focal imaging plane to fluoresce and produce induced light which passes maximally through the proximal collection aperture. Such wavelength shifting by dyes or phosphors allows for background signal from backscattered radiation to be reduced.

The first focal plane may be an input to a laser detector, a laser emitter, or an input to a diode illuminator source. The said second focal plane may be an input to an image intensifier, or an input to a CCD camera detector.

Of particular advantage, some embodiments of the present invention provide a lens and system wherein the coupling method includes transfer optics selected and arranged so as to achieve an improvement in transfer efficiency of at least 2-fold, as compared to a transfer efficiency of a system in the absence of said transfer optics, said improved efficiency of delivery at least in part a result of said improved transfer efficiency. In this case, the improved transfer efficiency is a result, at least in part, of the use of catadioptric lens configuration which allows for low f-number lenses and close-in use.

In some embodiments the transfer optics includes at least one collimation lens. In some embodiments, at least one of the collimation lenses is integral to the bulb of the lamp, and the collimation lens arranged to have its proximal surface located no more than 10 mm from the source of light within the light source. The transfer optics may include at least one refocusing lens. Coupling means can include an optical fiber, and the refocusing lens is focused at an illumination end of said optical fiber, or it may include optical coupling fluid, free space transmission, lenses, or other coupling means.

In some embodiments, a broadband light source is used. The broadband light source is fiber-coupled to the target region, and the coupling means includes at least one thermally-insulating optical fiber, the fiber is proximally optically coupled to the source and distally optically coupled to the target region. Alternatively, the source comprises a halogen recycle filament bulb lamp, and the coupling means comprises a collimating lens optically coupled to the filament and a reverse beam expander optically coupled to the collimating lens. The collimating lens may be integral to the bulb of the lamp and arranged to have its proximal surface located no more than 10 mm from the filament of the lamp, and the reverse expander is further optically coupled to the optical fiber, the source and coupling means selected and arranged so as to deliver a usable optical density of at least 25 mW/mm² into said fiber with negligible transferable thermal load.

One advantage of the various embodiments is that the arrangement provides for a flexible platform for regulatory approval. The high-density light provided by the instant invention can be delivered to the imaging lens using an insulating optical fiber, allowing for changing of illumination sources and wavelengths. This allows the lens to be used with multiple different sources of optical contrast.

In one aspect, the present invention provides a method of obtaining an image of a subject, said method comprising: projecting light to a subject using an illumination element; and obtaining an image of said subject by collecting light from said subject with a light collection element, wherein said light collection element comprising a catadioptric lens, and wherein said illumination element and said light collection element are arranged so as to provide a spatially co-registered illumination and imaging plane. In some embodiments the illumination element and said light collection element are arranged to provide collinear illumination.

### III. Medical Applications

In one aspect of the present invention, the lens and system is configured so as to be reversibly optically coupled to a medical probe.

In yet another aspect of the present invention, the lens and system incorporated into the body of a medical probe or system. Additionally, the lens and system is further incorporated into the patient end of a medical probe. Suitable medical probes include, but are not limited to: catheters, sheaths, guidewires, needles, trocars, surgical instruments, injection probes, and the like. Thus, the present invention may be configured as a medical probe and/or as a medical system.

In another aspect of the present invention, a medical illuminator catheter is provided. The medical illuminator catheter includes a biocompatible catheter sheath having a monitor end, a central body, and a patient end. A mixed optical and electrical connection plug is provided where the plug is located at or near the monitor end of said catheter. An integral broadband light source is provided for illuminating a target region and a collimating transfer lens for optically coupling said source to said region over free space. The source and lens are integrated into the body of the catheter, and the source and lens are further selected and arranged so as to deliver to the region an optical density of at least 1 mW/cm² over the target region with a transferable thermal load of no more than 100 mW heat per mW usable optical power delivered. An optical collection fiber is also provided for collecting light scattered from the target region and for transmitting the collected light from the patient end of the catheter, along a length of the catheter, to a connection plug at or near the monitor end of the catheter. Power supply wires are connected for transmitting electrical power to the light source. The wires typically traverse a length of the catheter and are electrically connected to both the light source and the connection plug.

In another aspect of the invention, the lens can be used as part of a system or method to perform surgery. For example, additional power can be provided on the illumination side of the device sufficient to perform activation of optically-activated drugs or compounds, or for performing ablation of the imaged tissue. Or, the lens can be used to generate real-time images of tissue, with optical or spectroscopic information overlaid on a video image from the lens (or hybridized with overlay and/or image co-processing on MRI images, CT images, ultrasound images, or other medical imaging device's image).

In one aspect, the present invention provides a method for performing surgery on a subject in need of surgery, said method comprising: collecting an image of a subject in need of surgery using an imaging system comprising a catadioptric lens; and performing surgery on said subject with the aid of said image. The subject can be an animal, preferably a mammal, such as a human being.

### IV. Exemplary Embodiments

One embodiment of the device will now be described in detail with reference to the Figures. While a specific embodiment is described and shown in the Figures, the invention is not limited to the specific configurations illustrated

Referring to FIG. 1, lens or imaging system 103 is illustrated in its component parts. Light emitted by a laser source (not shown) enters system 103 via fiber optic 113 connected to photon injection port 117. These photons emerge out of illumination aperture 159, slightly expanding to allow for beam expansion to match the working distance of the lens from the tissue to the size of the field. In this way, a 0.50 m distance gives a 10 cm wide illumination spot, while a 1.00 m distance gives a 20 cm wide illumination spot. Optionally, the relative size of this spot can be adjustable using adjustable focusing optics (shown only for the imaging portion of the lens system in this example).

Light ray 203 returning from the subject enters proximal aperture 205, is refracted posteriorly by lens 219, reflected anteriorly by first mirror 226, reflected posteriorly by second mirror 229, and then passes through hole 233 in mirror 226. Directly behind mirror 226, light ray 203 passes through bandpass filter 243, and finally through camera mount 247, where the image would strike a CCD imaging element, if present. Of note, light rays from different angles of emission or from different areas of the tissue are parallel after hole 223 until at least when passing mount 247. For this reason, filter 243 can be an angle-dependent interference or notch filter. An elegant solution, incorporated into the early systems we constructed, is to incorporate a tunable filter, such as the electrically tunable filter from CRI (Varispec filter, Cambridge Research Instruments, Inc., Woburn Mass.). In other designs, this parallel-ray region can be placed nearer the first lens, provided that the light rays strike the filter at a constant angle for all light rays.

In order to achieve focus, different methods can be used. In this example, the housing 107 for lens system 103 is divided into two halves, proximal tube 271 and distal tube 275, joined at sliding joint 278. Movement of the two halves 271 and 275 relative to each other is controlled in this example by motor 283, allowing for autofocus or distance-measuring software to control the lens focus.

Optionally, polarizing filters 292 and 294 can be placed in parallel or with crossed-axes, in order to select or exclude specularly reflected light, respectively, based upon retention or loss of polarization.

Lens system 103 can optionally have an integrated light source, such as a diode laser, rather than a fiber-coupled connection to a laser.

Operation of the device may now be described.

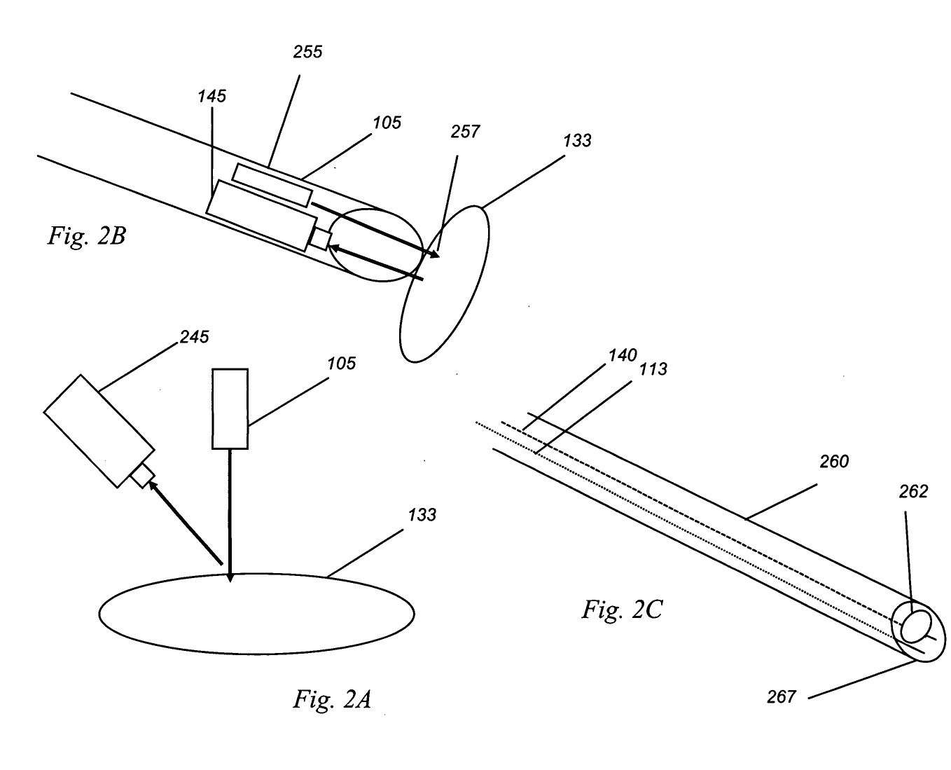

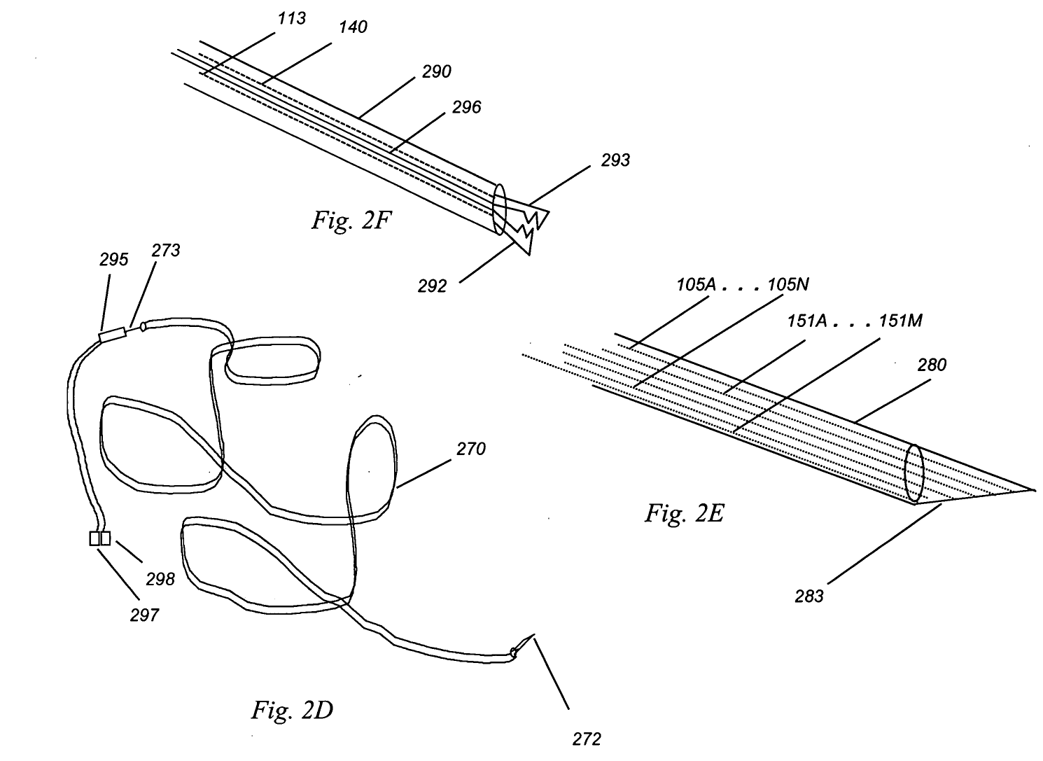

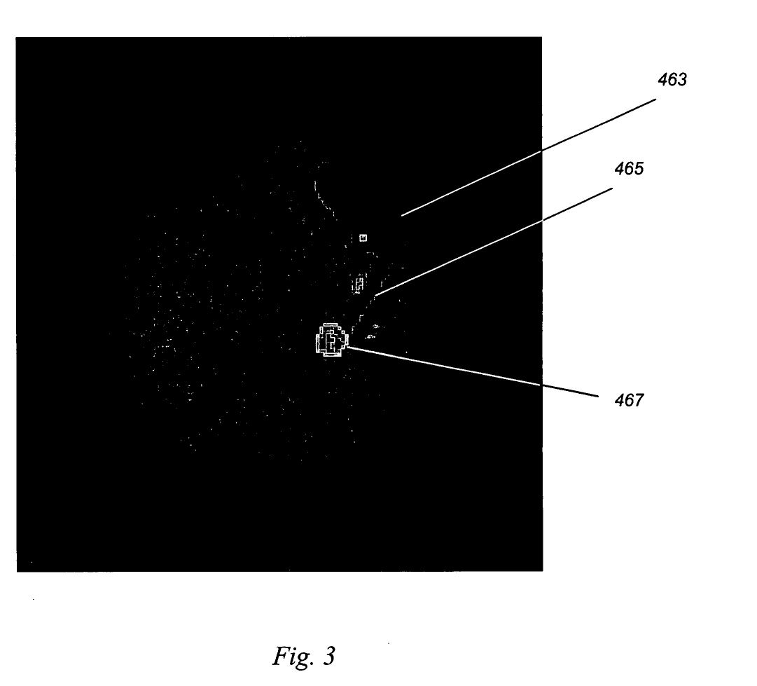

Lens system 103 may be incorporated into various medical devices, as shown in FIGS. 2A to 2F. This system may include an overhead surgical imager, including lens system 103, laser diode light source 105, and any required computer control (not shown).

Referring to FIGS. 2A to 2F, another aspect of the present invention is illustrated where the imaging system is incorporated into a medical device, or additionally a medical system. The medical device may be noninvasive or invasive. Examples of noninvasive systems according to embodiments of the present invention are shown in FIGS. 2A and 2B. In the exemplary embodiments a light emitter is preferably a laser 105. Detector 145 and laser 105 may be placed at a distance from the subject 133 of interest. In one embodiment the detector 145 is comprised of an imaging camera 245. Alternatively, laser 105 or detector 145, or both, may be assembled into a medical or surgical probe, such as but not limited to probe 255 shown in FIG. 2B. Probe 255 is preferably placed in contact with a surface 257 of the subject 133, such as in contact with the surface of tissue in a tissue region of interest.

In other embodiments, the imaging system of the present invention is incorporated into an invasive medical device or system. An invasive device may be broadly defined as when emitting elements, detecting elements, or both, are incorporated into a medical probe, needle, catheter, or the like, which is then used internally within a body. In such systems it may be important to stabilize optical fibers with respect to the tissue of interest in order to assist in measurement reproducibility. Examples of invasive systems according to embodiments of the present invention are illustrated in FIGS. 2C to 2F.

FIG. 2C shows a needle 260 with emitter fiber as coupler 113, detector fiber as coupler 140, injection port 262, and cutting edge 267. FIG. 2D shows a catheter 270 with extendable needle 272 controlled by wire 273, syringe attachment port 295, and with emitter fiber 297 and detector fiber 298 embedded into the needle (in a manner similar to that shown for needle 260). Another embodiment is shown in FIG. 2E, a scalpel 280 with multiple emitter fibers 105A to 105N and multiple detector fibers 151A to 151M embedded into cutting edge 283 of scalpel 280. In yet another embodiment, nibbler 290 is illustrated in FIG. 2F having emitter fiber as coupler 113 and detector fiber as coupler 140 embedded into jaws 292 ad 293, respectively, for monitoring and removing contrast-labeled tissue. Multiple emitter and detector fibers can be bundled to produce an optical imaging element that couples an imaging camera, such as imaging camera 245 shown in FIG. 2A, directly to the tissue, thus allowing much greater coupling efficiency that is achievable using a lens at a distance. An external imager may also be employed to monitor ongoing surgical procedures in real time. Additionally, an external surface probe may be used, for example, just prior to lymph node resection, to help identify the site of an initial incision, resulting in a smaller scar and less morbidity. Further, an internal needle or catheter may provide feedback to invasive or minimally invasive tools used to locate and treat a medical condition.

Of note, when light from a noninvasive or invasive system penetrates into tissue, the photons traveling between the light source and the light detector take a wide range of paths. The present device takes advantage of this effect as the scattering provides an averaging and volume sampling function. When detected illumination is measured after it has propagated through the tissue over substantially non-parallel multiple courses taken through the tissue between the time the photons are emitted and then detected, many regions of the tissue can be sampled, not merely the tissue on a narrow line between emission and detection. This allows a small but important feature, such as a the ability to sample the subsurface capillary layer of gastrointestinal mucosa, even if the probe itself is placed 1 cm from the intestinal wall.

In this embodiment, light source 105 of lens system 103 is a pulsed laser. However, source 105 may just as easily be any broadband LED, or be a polymer plastic that emits light under the influence of electrical power, or be a laser with broadening of the waveband via the input fiber impregnated with fluorescent dye, and so on, provided that source 105 meets the technical requirements of the improved lens system disclosed herein.

### EXAMPLES

The breadth of uses and advantages of the present invention are best understood by example, and by a detailed explanation of the workings of a constructed apparatus, now in operation and tested in model systems, animals, and humans, some of which are provided below. These and other advantages of the invention will become apparent when viewed in light of the accompanying drawings, examples, and detailed description. These examples are by no means intended to be inclusive of all uses and applications of the apparatus and are not intended to limit the scope of the invention in any way, but merely to serve as case studies by which a person, skilled in the art, can better appreciate the methods of utilizing, and the scope of, such a device.

### Example 1

### Improved Light Collection Using the Present Invention

One method to improve imaging efficiency is to improve the light collection efficiency—that is, to increase the fraction of target signal that reaches a detector.

In order to evaluate the impact of design considerations, we modeled the expected improvement in the efficiency of light collection achieved by altering various aspects of a collection lens design, and we then verified our model data using experiments. This model incorporated known features of light generation from tissue, the transmission characteristics of certain optical elements, and the like, and each aspect of the model was tested in the laboratory to confirm agreement of the predictions to those of measured results.

For this modeling, we assume that the lens focuses at 1 m with a field of view of 10-20 cm, and that the light source is a point of light, infinitesimally small, at the center of the imaging field. The highest theoretical limit for light throughput to a detector would be to place the detector directly against the tissue. In this case, all light leaving the tissue over a half-sphere of 2π solid-angle would strike the detector (those leaving the tissue surface and radiating inward do have a chance of scattering back out, but this does not change the overall ratio of lens efficiencies, and is therefore omitted in this example). This condition is set as 100% efficiency. Of course, flattening a living subject over a large region is impractical in many cases, and therefore a lens is used to image the region and to transfer this image to a detector.

First, we considered a better quality Nikon lens. The lens aperture forms a large circular target, measuring 10 mm in diameter, which intercepts light emerging in a cone from the tissue. This cone represents a fraction of the full 2π solid-angle. As an approximation, the surface area of the lens divided by the surface area of the half-sphere at the distance of the lens from the tissue provides a first-approximation of the total light collecting ability of the lens. The intercept area of the lens is then πr²=π* (10 mm)², or 314 mm². The area of the half sphere is given by (4/3)πr³=(4/3)*π* (1000 mm)³=1.26×10⁷ mm², for a collection ratio of (314/1.26×10⁷)=0.0025% effective light collection based upon aperture considerations alone. Lens throughput reduces this by a small amount, depending upon wavelength, lens materials, and lens coatings.

Now consider a catadioptric lens. The lens aperture is large, measuring 150 mm, for an aperture of 70,600 mm². The half-sphere is the same, for an effective light collection efficiency of 0.44%, an improvement of 178× based on aperture considerations alone.

These values were confirmed by experiment, with images collected such as dye in a tube in room light, as shown in FIG. 3. Here, dye 467 in tube 465 is seen in room light, while no signal is seen from other biological tissue such as hand 463. This improved collection has multiple advantages. First, while the lens design was optimized to allow for collinear illumination, this design can work quite well for systems which do not require illumination, such as luciferase-based systems.

Second, if the system were to be used in an operating room, a single lens system now allows one-handed operation in which the surgeon can point both the light source and the detection area with a single combined unit.

### Example 2

### Animal Imaging

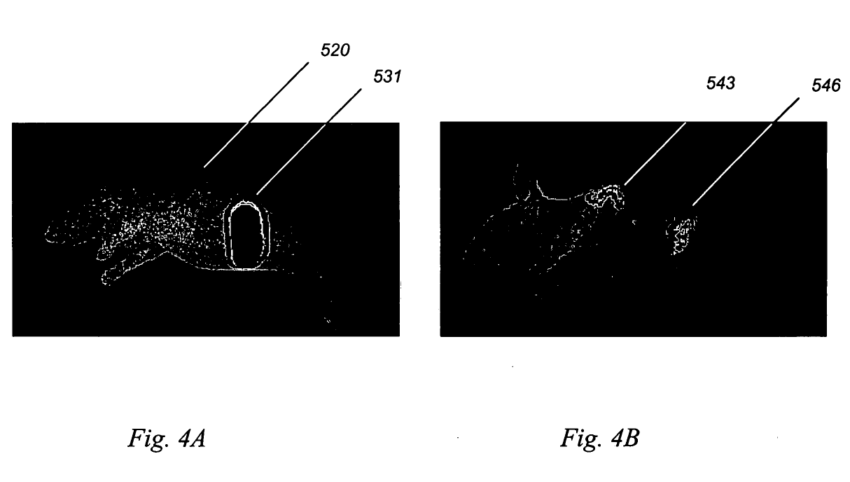

In order to test the validity of the data generated using the model shown in Example 1, we constructed a working system for experimental tests, under U.S. Government support. This system was used to image dye in animal, as shown in FIG. 4. In FIG. 4A, mouse 520 can be seen from room light, while dye 531 contained inside mouse 520 is seen. In FIG. 4B, dye is seen at sites 543 and 546.

This demonstrates that the system is operative and functional in its intended use, namely room light imaging of dyes within living animals.

This type of lens can be used in targeted surgery, as is disclosed in WO 2000/68665, incorporated by reference in full into this disclosure. One example is mounting the camera over an auxiliary dissection for breast cancer to look at sentinel lymph nodes. In this case, the dye could be injected into the skin, and the fluorescence imaged with the catadioptric lens and illumination system. The detection in this case could be from pulsed laser, frequency domain modulation with imaging of phase shifts, time domain pulsed sources with imaging of shifts in peak or first detected light images to determine dye half-life or lifetime shift, or even depth of the signal by depth-related color spectrum or peak time delay shifts, as is disclosed in WO 1993/013395 and elsewhere. Such modifications fall within the scope of the present invention when collected through a coregistered illumination and high efficiency collection lens.

Further, optical images and data from this lens may classified by tissue type, as disclosed in U.S. Pat. No. 5,987,346, also incorporated in full by reference, such that tissue is identified as containing fat, water, hemoglobin, bilirubin, beta carotene, metHemoglobin, carboxyhemoglobin, or other compounds. Such tissue data allows classification of tissue by type (skin, muscle, liver), by state (alive, dead, ablated, frozen, normal), or by a complex measure of metabolic and compositional states (breast cancer, breast cyst, normal breast ductal tissue). On could even process the returning data in a manner used to analyze pulse oximetry, such as is disclosed in U.S. Pat. No. 5,337,745, incorporated herein by reference in its entirety, to perform multispectral analysis of the blood or tissue. Such modifications also fall within the scope of the present invention when collected through a coregistered illumination and high efficiency collection lens.

In summary, improved imaging lenses with embedded, integral, and collinear illumination capacity has multiple expected and unexpected advantages. In certain applications, such as medical applications, this improvement enables real-time scans not previously possible due to light losses. We show that an improved imaging lens can be achieved by catadioptric configuration and integrated collinear illumination optics, which results in higher throughput, lowered cost and weight, and improved ease-of-use. Such improved lens systems may be designed as a standalone device, or embedded into an imaging system.

We have discovered an improved lens system for biomedical optics imaging applications for collecting light from tissue with an improved efficiency and geometry, and for delivering collinear, pre-aligned illumination to a the sample, for the purpose of enabling imaging applications. An illuminator lens has been constructed and tested, in which a catadioptric lens and mirror system is used for light collection, and integrated collimating illumination optics have been provided, has been constructed in accordance with the present invention to allow for high-efficiency light collection in operating room and radiology suite geometries. The efficient collection of light, and the aligned, collinear illumination, allows this lens system to operate at higher speeds and with improved ease-of-use. Scattered light, returning from the sample, is optionally collected by a lens and mirror system with integrated source optics. A movable, tunable, or adjustable filter system in line with the image allows spectroscopic analysis, such as multispectral or hyperspectral imaging, to be performed. A medical system incorporating the improved device, and medical methods of use, are described. This device has been built and tested in several configurations in models, animals, and humans, and has immediate application to several important problems, both medical and industrial, and thus constitutes an important advance in the art.

While the invention has been described in connection with specific embodiments it is evident that many variations, substitutions, alternatives and modifications will be apparent to those skilled in the art in light of the foregoing description and teaching. Accordingly, this description is intended to encompass all such variations, substitutions, alternatives and modifications as fall within the spirit of the appended claims.

What is claimed:

1. A medical imaging system, comprising:
an illumination element; and
a light collection element,
wherein said illumination and collection elements are arranged so as to provide a substantially coregistered illumination and imaging plane.

2. The imaging system according to claim 1, wherein said illumination element and said light collection element are arranged to provide collinear illumination and light collection.

3. The imaging system according to claim 1, wherein said illumination element and said light collection element together comprise a catadioptric lens and illumination system.

4. The imaging system according to claim 1, wherein said illumination element comprises:
a light input port; and
an illumination aperture optically coupled to said light input port.

5. The imaging system according to claim 4, wherein said illumination aperture is optically coupled to said light input port through a beam expander.

6. The imaging system according to claim 4 further comprising a light source to provide light through said light input port.

7. The imaging system according to claim 6, wherein said light source is a laser light source.

8. The imaging system according to claim 1, wherein said light collection element comprises:
a proximal entrance aperture;
a proximal lens;
a first mirror optically coupled to said entrance aperture through said proximal lens;
a second mirror optically coupled to said first mirror; and
a distal exit aperture optically coupled to said second mirror.

9. The imaging system according to claim 8 further comprising a first polarizing filter and a second polarizing filter.

10. The imaging system according to claim 9, wherein said first polarizing filter and said second polarizing filter are placed in parallel.

11. The imaging system according to claim 9, wherein said first polarizing, filter and said second polarizing filter are placed with cross-axis.

12. The imaging system according to claim 8 further comprising a bandpass filter.

13. The imaging system according to claim 8 further comprising a camera mount.

14. The imaging system according to claim 13, wherein a CCD camera is mounted on said camera mount.

15. The imaging system according to claim 8 further comprising a housing, wherein said housing comprises a proximal half and a distal half joined at a sliding joint.

16. The imaging system according to claim 8 further comprising an angle-dependent filter.

17. The imaging system according to claim 16, wherein said angle-dependent filter is an interference filter.

18. The imaging system according to claim 16, wherein said angle-dependent filter is a notch filter.

19. The imaging system according to claim 1, wherein said light collection element surrounds at least a portion of said illumination element.

20. The imaging system according to claim 19, wherein said illumination element comprises a central tube, wherein said light collection element is at least partially shielded from said illumination element by said central tube.

21. The imaging system according to claim 1, wherein said system is configured to be reversibly optically coupled to a medical probe.

22. The imaging system according to claim 1, wherein said system is incorporated into a body of a medical probe.

23. The imaging system according to claim 22, wherein said medical probe is selected from any one or more of: catheters, sheaths, guidewires, needles, trocars, surgical instruments, and injection probes.

24. The imaging system according to claim 1 wherein said system is incorporated into a medical system.

25. A method for obtaining an image of a sample, comprising: collecting an image of a sample using an imaging system comprising a catadioptric lens, wherein an illumination of the sample is effectively coregistered with the imaged view of the sample.

26. A method of obtaining an image of a subject, said method comprising:
projecting light onto a subject using an illumination element; and
obtaining an image of said subject by collecting light from said subject with a light collection element,
wherein said light collection element comprising a catadioptric lens,
and wherein said illumination element and said light collection element are arranged so as to provide a spatially co-registered illumination and image.

27. The method according to claim 26, wherein said illumination element and said light collection element are arranged to provide collinear illumination.

28. The method according to claim 26, wherein said illumination element and said light collection element together comprise a catadioptric lens and illumination system.

29. The method according to claim 26, wherein said light collection element further comprises a tunable bandpass filter.

30. A method for performing surgery on a subject in need of surgery, said method comprising:
collecting an image of a subject in need of surgery using an imaging system comprising a catadioptric lens; and
performing surgery on said subject with the aid of said image.
